(19) United States
(12) Patent Application Publication
(10) Pub. No.: US 2013/0211273 A1
(43) Pub. Date: Aug. 15, 2013

(54) METHOD AND APPARATUS FOR HEART RATE MEASUREMENT

(71) Applicants: Liang-Gee CHEN, Taipei (TW); Yu-Hsin Chen, Taipei (TW); Hong-Hui Chen, Taipei (TW)

(72) Inventors: Liang-Gee CHEN, Taipei (TW); Yu-Hsin Chen, Taipei (TW); Hong-Hui Chen, Taipei (TW)

(73) Assignee: National Taiwan University, Taipei City (TW)

(21) Appl. No.: 13/552,884

(22) Filed: Jul. 19, 2012

(30) Foreign Application Priority Data
Feb. 12, 2012 (TW) 101104499

(51) Int. Cl. A61B 5/0245; A61B 7/00

(52) U.S. Cl. 600/528

(57) ABSTRACT

The present invention provides a method for heart rate measurement, comprising the steps of: receiving a current heart sound signal from a channel; processing the current heart sound signal to be a pre-processed heart sound signal; receiving a template signal of a template database obtained independently from the current heart sound signal; and calculating a conformity between the pre-processed heart sound signal and the template signal to obtain a heart rate signal representing the pre-processed heart sound signal.

### BACKGROUND OF THE INVENTION

1. Field of the Invention

The present invention relates to a method for heart rate measurement, and more particularly, to a method for heart sound based heart rate measurement.

2. Description of the Prior Art

Heart rate is one of the basic yet important cardiovascular parameters. It has been used from the monitoring of daily living activities to the prediction of acute coronary event. Conventional methods for heart rate measurement are through electrocardiogram recording devices, pulse oximetry sensor or blood flow pulse sensor. Nevertheless, direct skin contact to these devices or the adhesive gel patch is required to get accurate records. Moreover, as these devices might not be easily obtained or accessed, their practicability is limited. Currently, the heart rate can also be measured by using a microphone to collect the sounds of the subject's heart beats and performing an algorithm to calculate the heart rate value. The algorithm, however, utilizes the matching of partial threshold values which is lack of self adaptive function, thus such method is not capable of outputting reliable heart rates on occasions where excess external interference exists or the subject is doing exercise.

Therefore, a need exists in the art for a method and an apparatus for heart rate measurement capable of outputting reliable heart rates over a wide range of variation in the heart rate or on occasions where excess external interference exists.

### SUMMARY OF THE INVENTION

The present invention provides a method for heart rate measurement, comprising the steps of: receiving a current heart sound signal from a channel; processing the current heart sound signal to be a pre-processed heart sound signal; receiving a template signal of a template database obtained independently from the current heart sound signal; and calculating a conformity between the pre-processed heart sound signal and the template signal to obtain a heart rate signal representing the pre-processed heart sound signal.

In the aforementioned method for heart rate measurement of the present invention, the channel is a microphone.

In the aforementioned method for heart rate measurement of the present invention, the template database is a cloud-based database comprising a plurality of template signals.

In the aforementioned method for heart rate measurement of the present invention, another template signal of the template database will be received when the conformity is lower than a predetermined value.

In the aforementioned method for heart rate measurement of the present invention, the step of processing the current heart sound signal to be a pre-processed heart sound signal is performed by a band-pass filter, a decimation filter, an absolute-value generator and a low-pass filter.

In the aforementioned method for heart rate measurement of the present invention, the step of calculating a conformity between the pre-processed heart sound signal and the template signal is performed by a vector distance calculating unit and a high conformity conversion unit.

In the aforementioned method for heart rate measurement of the present invention, the template signal has a length in a range between about 0.7 second and 1.0 second.

The present invention further provides an apparatus for heart rate measurement, comprising: a heart sound signal processor configured to receive a current heart sound signal from a channel and process the current heart sound signal to be a pre-processed heart sound signal; and a conformity calculating device configured to receive a template signal of a template database obtained independently from the current heart sound signal and calculate a conformity between the pre-processed heart sound signal and the template signal to obtain a heart rate signal representing the pre-processed heart sound signal.

In the aforementioned apparatus for heart rate measurement of the present invention, the channel is a microphone.

In the aforementioned apparatus for heart rate measurement of the present invention, the template database is a cloud-based database comprising a plurality of template signals.

In the aforementioned apparatus for heart rate measurement of the present invention, another template signal of the template database will be received when the conformity is lower than a predetermined value.

In the aforementioned apparatus for heart rate measurement of the present invention, the heart sound signal processor comprises a band-pass filter, a decimation filter, an absolute-value generator and a low-pass filter.

In the aforementioned apparatus for heart rate measurement of the present invention, the conformity calculating device comprises a vector distance calculating unit and a high conformity conversion unit.

In the aforementioned apparatus for heart rate measurement of the present invention, the template signal has a length in a range between about 0.7 second and 1.0 second.

### BRIEF DESCRIPTION OF THE DRAWINGS

FIG. 1 is a block diagram of an apparatus for heart rate measurement in accordance with a first embodiment of the present invention.

FIG. 2A illustrates the original heart sound signal from the microphone.

FIG. 2B illustrates the pre-processed heart sound signal obtained after the processing measures have been taken.

FIG. 3 illustrates the similarity-level trace.

FIG. 4 illustrates the feature of high similarity patterns (HSP) in the similarity-level trace.

### DETAILED DESCRIPTION OF THE PREFERRED EMBODIMENTS

The detailed embodiments of the present invention will be provided in the following paragraphs. It is to be noted that the embodiments of the present invention are exemplary. The present invention is not limited to the embodiments comprising specific features, structures or properties and the scope thereof is defined by the appended claims. In addition, the drawings do not specifically illustrate all unnecessary features of the present invention. For those illustrated in the drawings, they may be represented in simplified form or schematic manner. Furthermore, for the sake of clarity, the sizes of the components may be magnified in the drawings or not in actual proportion. Whether or not the components are simplified in form or the features are illustrated in detail, they fall within the scope of knowledge of the art so that they can be implemented by those skilled in the art in view of other embodiments related to the features, structures or properties.

The present invention utilizes a high sensitivity microphone to measure the sounds of the subject's heart beats so as to calculate the subject's heart rate. The present invention utilizes the matching of the current heart sound signal measured from the subject and the template signal obtained independently from the current heart sound signal to overcome the drawback of inaccurate calculation results which may be caused by excess external interference or a wide range of variation in the heart rate. The apparatus and method for heart rate measurement of the present invention will be detailed in the following paragraphs.

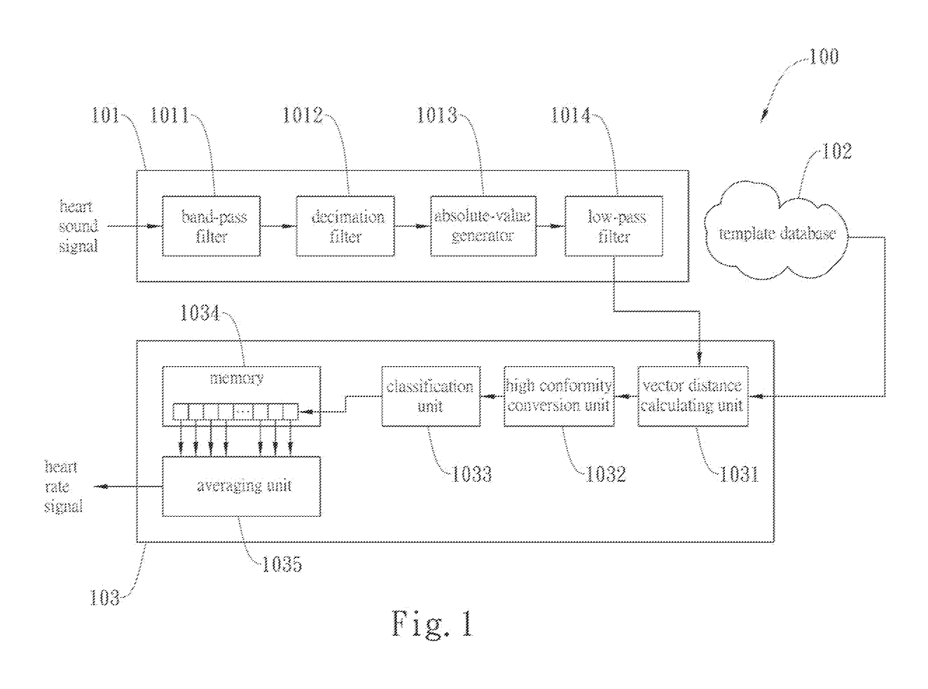

FIG. 1 is a block diagram of an apparatus for heart rate measurement in accordance with a first embodiment of the present invention. The apparatus 100 for heart rate measurement of the present invention comprises a heart sound signal processor 101 comprising a band-pass filter 1011, a decimation filter 1012, an absolute-value generator 1013 and a low-pass filter 1014, and a conformity calculating device 103 comprising a vector distance calculating unit 1031, a high conformity conversion unit 1032, a classification unit 1033, a memory 1034 and an averaging unit 1035.

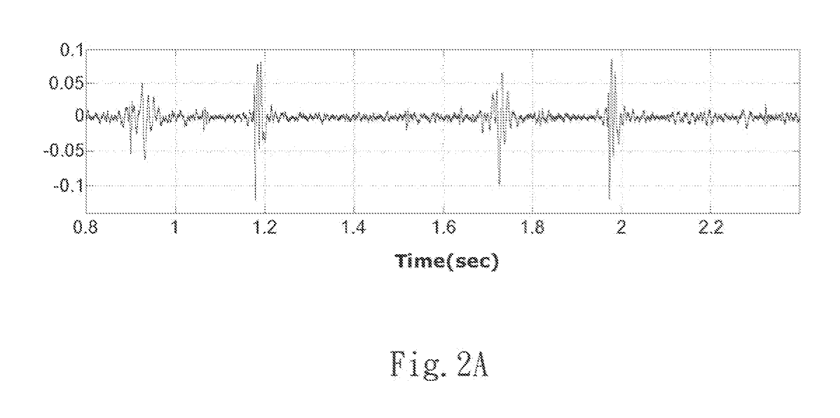

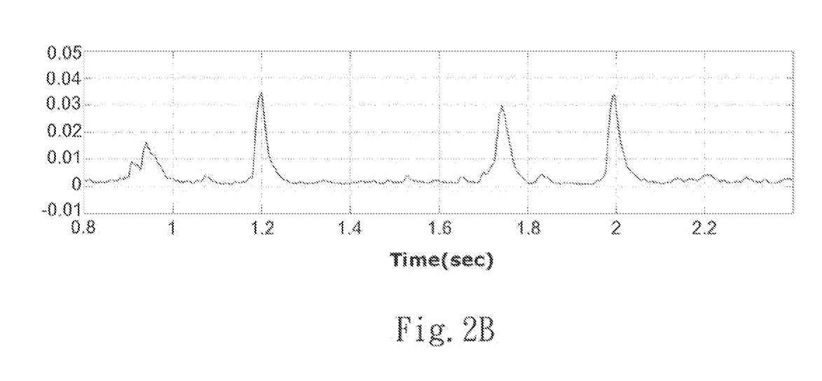

The apparatus 100 for heart rate measurement of the present invention is configured to receive a heart sound signal from a microphone as shown in FIG. 2A. FIG. 2A illustrates the original heart sound signal from the microphone. To avoid and eliminate the effects brought by noises coupled into the heart sound signal, the following processing measures are required to obtain a pre-processed heart sound signal. First, the apparatus 100 for heart rate measurement of the present invention filters the heart sound signal through the band-pass filter 1011 with cutoff frequencies at 80 Hz and 150 Hz. Then, the signal is down-sampled to the sampling frequency of 500 Hz through the decimation filter 1012. Next, the signal is full-wave rectified through the absolute-value generator 1013, and is further filtered using the low-pass filter 1014 with cutoff frequency at 15 Hz to obtain a pre-processed heart sound signal. FIG. 2B illustrates the pre-processed heart sound signal obtained after the processing measures have been taken. As can be seen from FIG. 2B, the high-frequency background noise is mostly suppressed

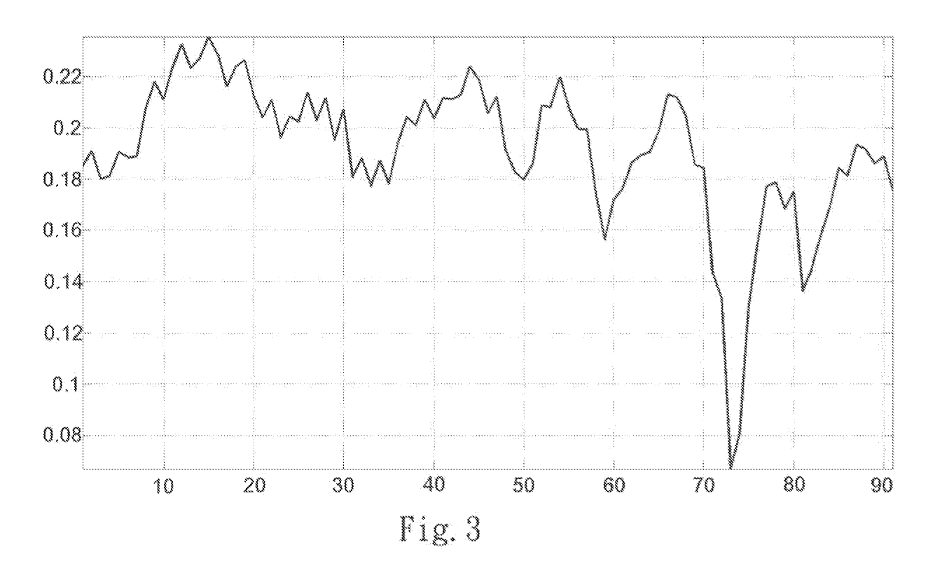

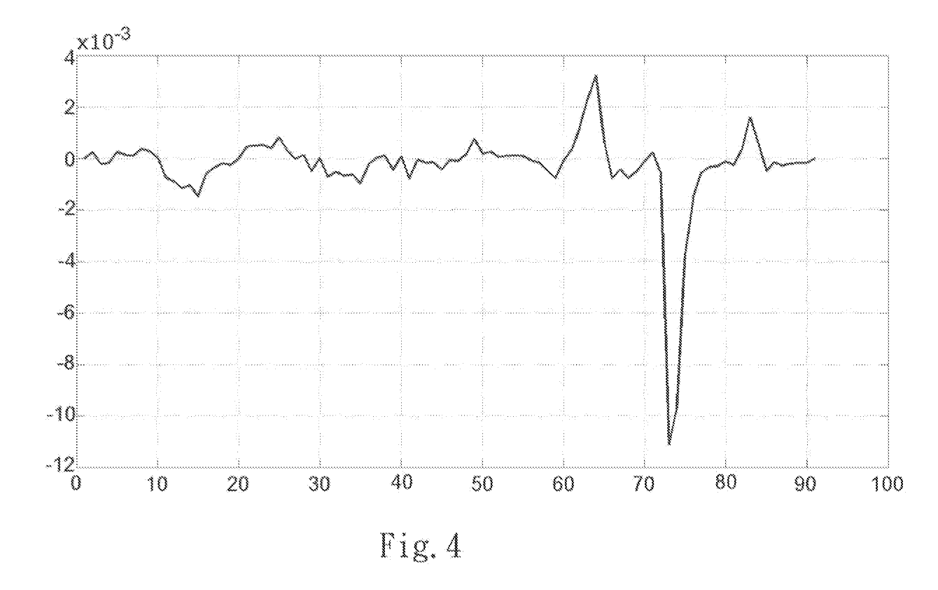

The pre-processed heart sound signal is sent to the conformity calculating device 103 to undergo the conformity calculating procedure. The conformity calculating device 103 is configured to receive the pre-processed heart sound signal and a template signal of a template database 102. The template signal has a length in a range between 0.7 second and 1.0 second. In an embodiment of the present invention, the template signal of the template database 102 needs to be set prior to the measurement of the subject's heart sound signal and can be stored in the cloud. For example, the template signal can be set without any external interference so as to obtain a correct template signal. Next, the apparatus 100 for heart rate measurement of the present invention utilizes the vector distance calculating unit 1031 to compare the pre-processed heart sound signal and the template signal. The length of the pre-processed heart sound signal block is the same as that of the template signal. The comparison is performed by calculating the distance between the vector of the template signal and the vector of the pre-processed heart sound signal block. The distances could be plotted as a similarity-level trace as shown in FIG. 3. The feature of high similarity patterns (HSP) in the similarity-level trace is then extracted by the high conformity conversion unit 1032 as shown in FIG. 4. Therefore, the lower values of FIG. 4 represent the locations and values where there is a high conformity between the pre-processed heart sound signal and the template signal. Next, the lower values are classified, marked as possible heart beat periods and fed to the memory 1034 by the classification unit 1033. Next, the aforementioned steps are performed again to obtain a new heart beat period. The heart beat periods obtained in a predetermined time period (e.g. 5 to 20 seconds) are averaged by the averaging unit 1035 to obtain the heart rate signal in this time period.

In an embodiment of the present invention, the template database 102 comprises a plurality of template signals, e.g. the subject's normal heart rate, fast heart rate, slow heart rate, etc., and the plurality of template signals are set prior to the measurement of the subject's heart sound signal. In the aforementioned process, if the conformity between the pre-processed heart sound signal and the template signal is found to be lower than a predetermined value, which is likely to occur, for example, when the measurement is performed while the subject is doing exercise and the template signal represents a normal heart rate, the apparatus 100 for measurement of the present invention will receive another template signal of the template database 102 and perform the matching again. Consequently, the present invention exhibits a stable function for calculating the heart rate.

The scope and spirit of the present invention are not limited to the aforementioned embodiments. In addition, it will be understood that the drawings are merely schematic representations of the invention and not illustrated according to actual scale, and some of the components may have been magnified or simplified for purposes of pictorial clarity. The embodiments depicted above and the appended drawings are exemplary and are not intended to limit the scope of the present invention. The scope thereof is defined by the appended claims.

What is claimed is:

1. A method for heart rate measurement, comprising the steps of:
receiving a current heart sound signal from a channel;
processing the current heart sound signal to be a pre-processed heart sound signal;
receiving a template signal of a template database obtained independently from the current heart sound signal; and
calculating a conformity between the pre-processed heart sound signal and the template signal to obtain a heart rate signal representing the pre-processed heart sound signal.

2. The method for heart rate measurement according to claim 1, wherein the channel is a microphone.

3. The method for heart rate measurement according to claim 1, wherein the template database is a cloud-based database comprising a plurality of template signals.

4. The method for heart rate measurement according to claim 3, wherein another template signal of the template database will be received when the conformity is lower than a predetermined value.

5. The method for heart rate measurement according to claim 1, wherein the step of processing the current heart sound signal to be a pre-processed heart sound signal is performed by a band-pass filter, a decimation filter, an absolute-value generator and a low-pass filter.

6. The method for heart rate measurement according to claim 1, wherein the step of calculating a conformity between the pre-processed heart sound signal and the template signal is performed by a vector distance calculating unit and a high conformity conversion unit.

7. The method for heart rate measurement according to claim 1, wherein the template signal has a length in a range between about 0.7 second and 1.0 second.

8. An apparatus for heart rate measurement, comprising:
a heart sound signal processor configured to receive a current heart sound signal from a channel and process the current heart sound signal to be a pre-processed heart sound signal; and
a conformity calculating device configured to receive a template signal of a template database obtained independently from the current heart sound signal and calculate a conformity between the pre-processed heart sound signal and the template signal to obtain a heart rate signal representing the pre-processed heart sound signal.

9. The apparatus for heart rate measurement according to claim 8, wherein the channel is a microphone.

10. The apparatus for heart rate measurement according to claim 8, wherein the template database is a cloud-based database comprising a plurality of template signals.

11. The apparatus for heart rate measurement according to claim 10, wherein another template signal of the template database will be received when the conformity is lower than a predetermined value.

12. The apparatus for heart rate measurement according to claim 8, wherein the heart sound signal processor comprises a band-pass filter, a decimation filter, an absolute-value generator and a low-pass filter.

13. The apparatus for heart rate measurement according to claim 8, wherein the conformity calculating device comprises a vector distance calculating unit and a high conformity conversion unit.

14. The apparatus for heart rate measurement according to claim 8, wherein the template signal has a length in a range between about 0.7 second and 1.0 second.
